(19) United States
(12) Plant Patent Application Publication
(10) Pub. No.: US 2004/0205869 P1
(43) Pub. Date: Oct. 14, 2004

(54) Strawberry variety named 'Sable'

(76) Inventor: Andrew R. Jamieson, Nova Scotia (CA)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/747,871

(22) Filed: Dec. 29, 2003

Related U.S. Application Data
(63) Continuation of application No. 09/935,478, filed on Aug. 23, 2001, now abandoned.

(51) Int. Cl. A01H 5/00

(52) U.S. Cl. PLT/208

(57) ABSTRACT

This invention relates to a new and distinct variety of strawberry (Fragaria xananassa) named ‘Sable’. The invention is an early season, short-day variety similar to ‘Veestar’. ‘Sable’ is distinguished from ‘Veestar’ by its higher productivity, larger fruit, broader fruit, and pattern of disease resistance.

### BOTANICAL CLASSIFICATION

Fragaria xananassa

### VARIETAL DENOMINATION

‘Sable’

### BACKGROUND OF THE INVENTION

The present invention includes a new and distinct cultivar of Fragaria xananassa known by the varietal name ‘Sable’, originally designated as “K90-1”. The new variety resulted from a controlled cross in an ongoing breeding program between the unpatented cultivar ‘Veestar’ and ‘Cavendish’ (U.S. Plant Pat. No. 11,110). ‘Sable’ was discovered as a seedling in a controlled breeding plot near Sheffield Mills, Nova Scotia at the Sheffield Farm, a field-station of the Atlantic Food and Horticulture Research Center in 1990, where it was selected and propagated asexually by stolons at the Atlantic Food and Horticulture Research Centre in Kentville. Asexual propagules from this original source have been produced annually in a greenhouse at the Atlantic Food and Horticulture Research Centre, Kentville, Canada. ‘Sable’ has been tested at the Atlantic Food and Horticulture Research Centre, Kentville, N.S. (starting in 1991) and also, research centres at Charlottetown, Prince Edward Island, Buctouche, New Brunswick, and Fredericton, New Brunswick, all of Canada and at the Newfoundland Department of Agriculture field site at Pynn's Brook, Newfoundland, Canada. This propagation and testing has demonstrated that the combination of traits disclosed herein which characterize the new variety are fixed and retained true to type through successive generations of asexual reproduction via stolons.

### DESCRIPTION OF THE DRAWINGS

FIG. 1 shows plant parts of the new variety, typical in size, shape, and color;

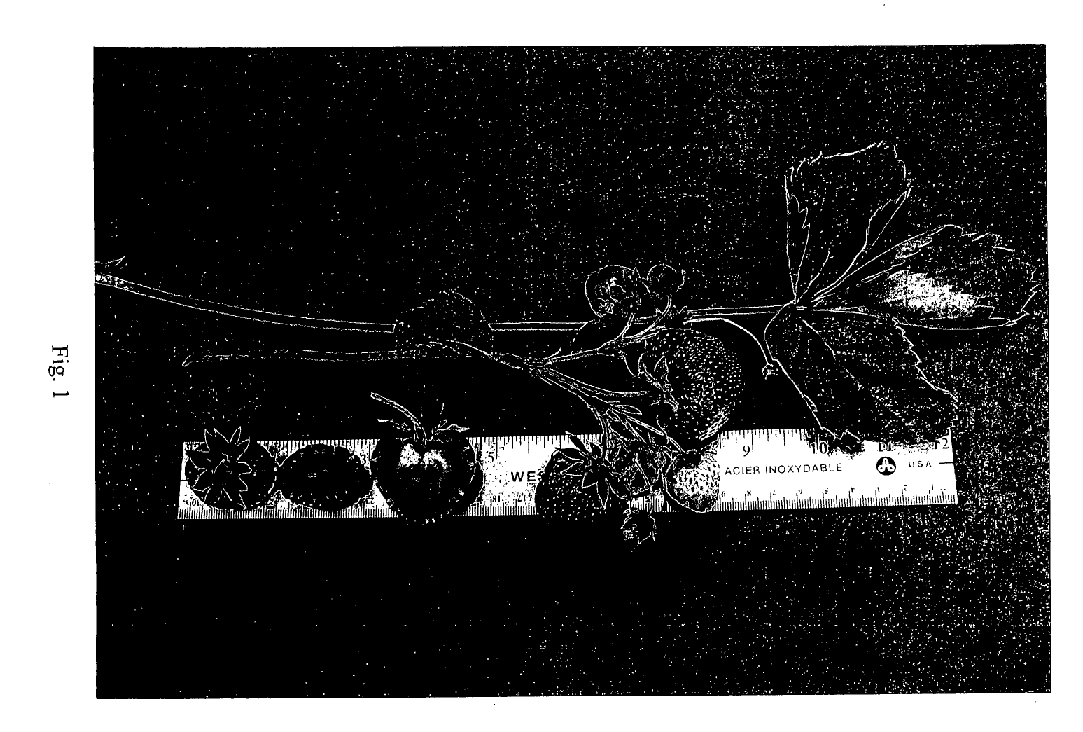

FIG. 2 shows the flowers of the new variety illustrating the ruffled appearance; and

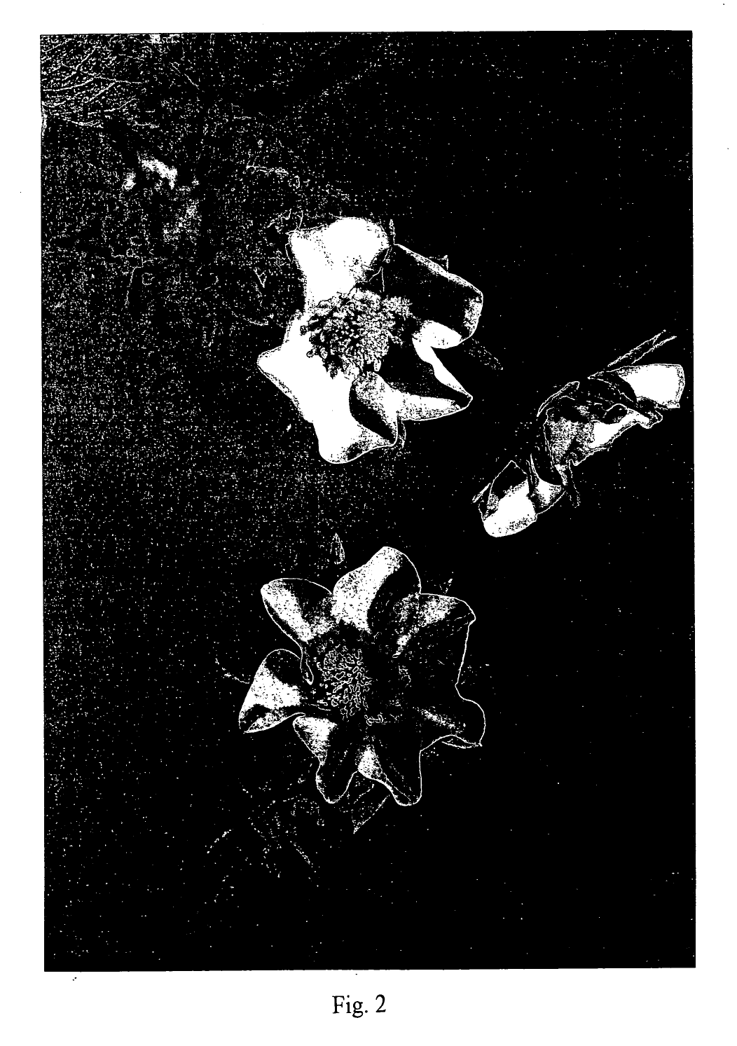

FIG. 3 shows the fruit of the new variety.

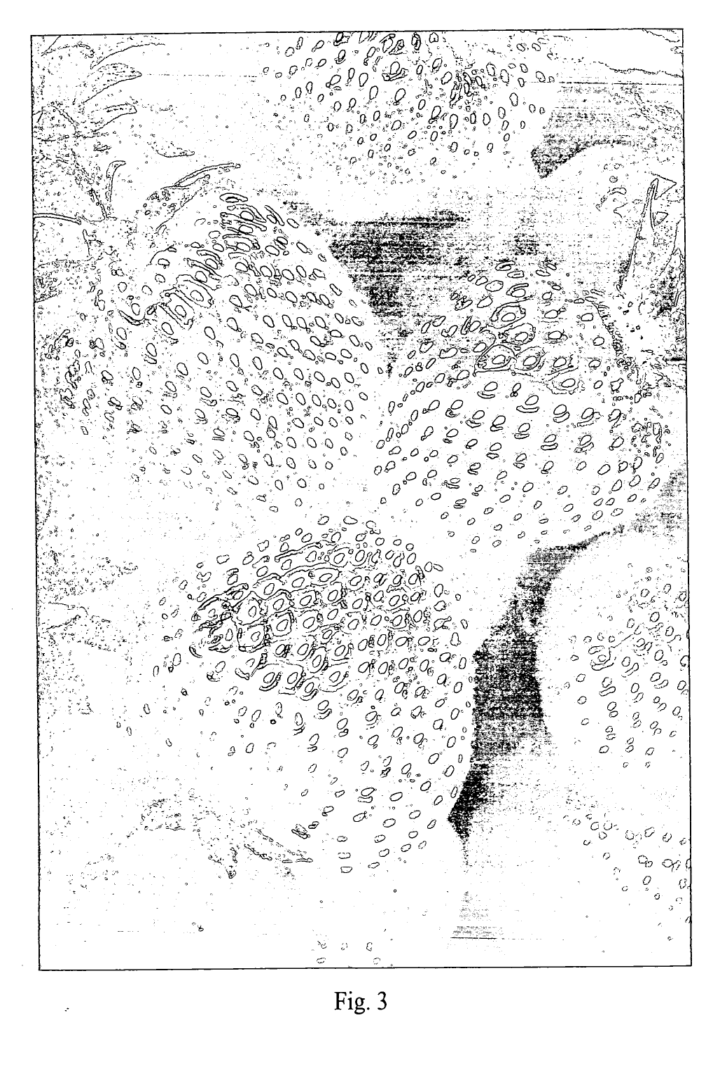

### DESCRIPTION OF THE PLANT

The following detailed botanical description of the new variety is based upon measurements and observations taken of plants and fruit grown in Kentville, Nova Scotia, Canada. Observations were taken from each variety as grown in a side-by-side field trial. This description is in accordance with UPOV terminology. Color designations, color descriptions, and other phenotypical descriptions may deviate from the stated values and description depending upon variation in the environment, seasonal, climatic, and cultural conditions, however, it is believed that this description will apply to the ‘Sable’ plants grown in similar conditions of soil and climate elsewhere. Colors are described using a standard R.H.S. Colour Chart. Descriptive information on the new variety is presented in Tables 1, 2, and 3. In the tables, the flowers described are secondary flowers. The fruit described is the secondary fruit of the maiden crop, thirteen or fourteen months after planting. The harvest data in Table 4 is based on a complete harvest of the crop. Principal differences between ‘Sable’ and the unpatented varieties ‘Annapolis’ and ‘Veestar’ are set forth.

Classification: The new variety is botanically classified as Fragaria xananassa and commercially classified as a short-day strawberry.

Plant and foliage: When propagated in the nursery, ‘Sable’ runners freely producing similar numbers of runners to ‘Annapolis’ and ‘Veestar’. The plants of ‘Sable’ are of medium density with globose habit and strong vigor. As shown in Table 1, leaf color of ‘Sable’, ‘Veestar’, and ‘Annapolis’ are Green Group 137A on the upper surface and lighter Green Group 137C on the under surface. ‘Sable’ tends to have smaller leaflets than ‘Veestar’ and ‘Annapolis’. ‘Sable’ leaflets have higher numbers of serrations than ‘Veestar’ and ‘Annapolis’. Leaflet serrations are semi-round for all three varieties. The venation of ‘Sable’ leaflets is pinnate. Leaf and petiole pubescence for ‘Sable’, ‘Veestar’ and ‘Annapolis’ are similar in density.

TABLE 1
Foliar characteristics for
‘Sable’, ‘Veestar’, and ‘Annapolis’

| | Cultivar | | |
|---|---|---|---|
| Foliar Character | ‘Sable’ | ‘Veestar’ | ‘Annapolis |
| Leaf color | Green Group | Green Group | Green Group |
| upper surface | 137A | 137A | 137A |
| lower surface | 137C | 137C | 137C |
| Central leaflet | | | |
| Length (mm) | | | |
| mean | 79.0 | 86.3 | 92.3 |
| range | 70-93 | 62-110 | 71-111 |
| Width (mm) | | | |
| mean | 63.9 | 68.0 | 73.4 |
| range | 55-77 | 50-85 | 60-93 |
| Length/width ratio | 1.24 | 1.27 | 1.26 |
| No. leaflets/leaf | 3 | 3 | 3 |
| Leaf convexity | cupped | cupped | cupped |
| Serrations | | | |
| number | many | moderate | moderate |
| size | small | medium | medium-large |
| shape | semi-round | semi-round | semi-round |
| tip serration size | small | medium | medium |
| Leaf pubescence | medium | medium | medium |
| Petiole pubescence | | | |
| density | sparse | sparse | sparse |
| direction | perpendicular | perpendicular | perpendicular |

Flower and fruit production characteristics: The length of bloom for ‘Sable’ is about three weeks when grown in Kentville, Nova Scotia, Canada in a matted row cultural system. Flowering for both ‘Sable’ and ‘Veestar’ typically begins on May 21 and ends on June 11. The length of bloom for ‘Annapolis’ is shorter, typically beginning on May 23 and ending by June 10. Flowers of ‘Sable’ and the reference varieties are white which is not included on the R.H.S. Colour Chart. The anther color is Yellow-Orange Group 17A. Flowers of ‘Sable’ and ‘Annapolis’ are positioned even with the foliar canopy but flowers of ‘Veestar’ are just beneath the canopy. Secondary flowers of ‘Sable’ are slightly larger than ‘Veestar’ and slightly smaller than ‘Annapolis’, on the average. Secondary flowers of ‘Sable’ and the reference varieties usually have 5 or 6 petals. Petals of ‘Sable’ are wider than long, in contrast to ‘Annapolis’ which are longer than wide. The flowers of the reference varieties are flat in appearance whereas those of ‘Sable’ appear moderately ruffled. The calyx of ‘Sable’ is larger than for the reference varieties. The inner calyx has the same diameter as the outer calyx. Trusses of ‘Sable’ are semi-erect at first picking but become prostrate during the harvest period. The size of the calyx in relation to fruit diameter is smaller for ‘Sable’ and ‘Annapolis’ whereas the ‘Veestar’ calyx is about the same diameter as the fruit. The position of the calyx in a basin to even for ‘Sable’ is in contrast to the other two varieties which have a position even with the top of the berry. Fruit of ‘Sable’ and ‘Annapolis’ are firmer than ‘Veestar’. Fruit of ‘Sable’ are more glossy in appearance that the reference varieties. As shown in Table 2, the fruit color of ‘Sable’ and ‘Annapolis’ are very similar; both varieties have a lighter red exterior and interior than ‘Veestar’. ‘Veestar’ has a smaller internal white area beneath the calyx. The achenes of ‘Sable’ are Green-Yellow Group 1A but darken to Orange-Red Group 34A when exposed to sunlight.

As used in Table 2, fruit firmness was recorded by penetration using an Ametek firmness tester with a 6 mm diameter v-notched probe inserted 6 mm into the fruit. The unit of measurement was the Newton, abbreviated ‘N’ in the Table. The unit “g” is used to illustrate the measure of force in grams to puncture the skin with a blunt insect pin with a diameter of 0.23 mm.

TABLE 2
Flower and fruit characteristics for
‘Sable’, ‘Veestar’, and ‘Annapolis’

| | Cultivar | | |
|---|---|---|---|
| Character | ‘Sable’ | ‘Veestar’ | ‘Annapolis’ |
| Flower position | even | beneath | even |
| (relative to leaf canopy) | | | |
| Flower truss length | medium-long | short-medium | medium |
| | 28.8 cm | 22.6 cm | 24.2 cm |
| Number of flowers/truss | 8.0 | 6.9 | 5.0 |
| Number of petals | 5.3 | 5.5 | 5.6 |
| Flower size (mm diameter) | 25.2 | 23.6 | 27.4 |
| Petal length (mm) | 9.6 | 9.2 | 11.0 |
| Petal width (mm) | 11.4 | 10.3 | 10.0 |
| Petal spacing | slightly | slightly | touching |
| | overlapping | overlapping | |
| Calyx size | | | |
| Inner calyx (mm diameter) | 24.6 | 21.2 | 18.9 |
| Outer calyx (mm diameter) | 24.2 | 19.7 | 17.3 |
| Calyx position | even to in a basin | even | even |
| Fruit stem length | medium | medium | medium |
| Fruit shape | | | |
| Length/width ratio | 0.78 | 1.08 | 0.87 |
| Subjective | cordate | conic to short-conic | short-conic |
| Seed position | slight indent | even | slight indent |
| Fruit firmness (N) | 3.0 | 2.8 | 3.0 |
| Skin toughness (g) | 11.6 | 8.4 | 9.1 |
| Color (R.H.S. Colour Chart) | | | |
| Calyx | Green Group | Green Group | Green Group |
| | 137C | 137C | 137C |
| Fruit exterior | Red Group | Red Group | Red Group |
| | 45A | near 46A | 45A |
| Fruit interior | Red Group | Red Group | Red Group |
| pith | 42B | near 46B | 42B |
| Cortex | 42A | near 46B | 42A |

Disease resistance: ‘Sable’ has a much higher level of resistance to red stele root rot (Phytophthora fragariae) than ‘Veestar’ and reaction to distinct races of pathogen are given in Table 3. Compared with ‘Annapolis’, ‘Sable’ is more resistant to C-1 while ‘Annapolis’ is more resistant to C-6. Resistance to race C-1 is rare among strawberry varieties (Advances in Strawberry Research 14:31-35). ‘Sable’ and ‘Annapolis’ are susceptible to powdery mildew (Sphaerotheca macularis) but ‘Veestar’ is moderately resistant. ‘Sable’ is moderately resistant common leaf spot (Mycosphaerellafragaria) but ‘Veestar’ and ‘Annapolis’ are susceptible. ‘Annapolis’ and ‘Veestar’ are less affected by fruit rot (Botrytis cinerea) than ‘Sable’.

TABLE 3
Resistance of ‘Sable’, ‘Veestar’, and ‘Annapolis’
to races of Phytophthora fragariae (red stele root rot)

| Canadian | American | Cultivar | | |
|---|---|---|---|---|
| race | race | ‘Sable’ | ‘Veestar’ | ‘Annapolis’ |
| C-1 | | I | S | S |
| C-2 | A-6 | R | S | R |
| C-3 | A-4 | R | R | R |
| C-4 | A-3 | R | S | R |
| C-5 | A-5 | S | S | S |
| C-6 | A-7 | S | R | R |
| C-7 | | S | S | S |
| C-8 | | S | S | S |

S = susceptible;
I = intermediate;
R = resistant

Production characteristics: ‘Sable’ has been widely tested for several years. Compared with the early season varieties ‘Veestar’ and ‘Annapolis’, ‘Sable’ produces higher yields, as shown in Table 4. The fruit of ‘Sable’ are slightly smaller (by weight) than ‘Annapolis’ but much larger than ‘Veestar’. ‘Sable’ has a similar percent marketability and season of harvest to the reference varieties. Subjectively, the fruit of ‘Sable’ are sweet and flavorful and preferred over the reference varieties.

The “Mean Harvest (day of year)” heading in Table 4 is used to indicate the day you are most likely to have the peak harvest. It is an average of each plot at each site and for ‘Sable’ was the 191.7^{th} day of the year in 1995.

TABLE 4
Performance of ‘Sable’ and ‘Veestar’ or ‘Annapolis’ for
1995, 1996, 1998, and 2000 averaged over several sites:
Kentville NS, Charlottetown PEI, Fredericton NB, and
Pynn's Brook Nfld. Plants were grown in matted rows
and three blocks of 3 m long rows were harvested at
each site.

| | Total yield | % yield | Size | Mean harvest |
|---|---|---|---|---|
| | (t/ha) | marketable | (g/fruit) | (day of year) |
| 1995 (four sites) | | | | |
| ‘Sable’ | 17.4 | 90.2 | 12.7 | 191.7 |
| ‘Veestar’ | 12.8 | 90.6 | 8.7 | 191.5 |
| 1996 (four sites) | | | | |
| ‘Sable’ | 13.5 | 86.7 | 9.0 | 192.8 |
| ‘Veestar’ | 12.8 | 89.8 | 7.0 | 193.3 |
| 1998 (one site) | | | | |
| ‘Sable’ | 19.7 | 87.3 | 11.7 | 180.3 |
| ‘Veestar’ | 12.0 | 92.5 | 8.0 | 178.3 |
| 2000 (three sites) | | | | |
| ‘Sable’ | 11.7 | 87.2 | 9.6 | 191.7 |
| ‘Annapolis’ | 7.3 | 93.2 | 11.9 | 192.8 |

I claim:

1. The new and distinct variety of strawberry plant named ‘Sable’ as described and illustrated.
